(19) United States
(12) Patent Application Publication
(10) Pub. No.: US 2023/0348861 A1
(43) Pub. Date: Nov. 2, 2023

(54) CULTURING OF STEM CELLS

(71) Applicant: REVATIS SA, Liège (BE)

(72) Inventors: Didier SERTEYN, Liège (BE); Justine CEUSTERS, Crisnée (BE); Hélène GRAIDE, Angleur (BE)

(21) Appl. No.: 17/800,719

(22) PCT Filed: Feb. 19, 2021
(86) PCT No.: PCT/EP2021/054116

§ 371 Date: Aug. 18, 2022

(30) Foreign Application Priority Data
Feb. 21, 2020 (EP) 20158846.4

(51) Int. Cl. C12N 5/0775; C12N 5/00; C12N 9/64

(52) U.S. Cl. CPC C12N 5/0668; C12N 5/0062; C12N 5/0068; C12N 9/6424; C12N 2500/34; C12N 2500/84; C12N 2513/00; C12Y 304/21062

(57) ABSTRACT

The present invention provides a novel culture medium and method for culturing stem cells, preferably muscle-derived mesenchymal stem cells. The culture medium comprises clotted blood plasma at 5-70% in base medium. The cultured cells may be released without use of trypsin by resolubilizing the clotted blood plasma with the assistance of proteolytic enzymes or anticoagulation agents.

### FIELD OF THE INVENTION

The invention relates to the culturing of stem cells, more specifically mammalian stem cells, including but not limited to mesenchymal stem cells, more specifically to culturing in a 3-D culture medium. More particularly, the invention relates to the culturing of mesenchymal stem cells (MSCs) such as MSCs derived from mammalian muscular tissue.

### BACKGROUND OF THE INVENTION

Cell culture comprises techniques that allow cells to grow outside their natural environment. Depending on cell type, cell proliferation will occur in suspension or by adhesion to a support thanks to adhesion factors added to the growing medium. Cell growth will take place thanks to a medium in which the cells are contained and which provides all nutritive elements required for the cell's wellbeing. The medium also comprises antibiotics, antifungal, and other components.

It is generally known that cell culture requires appropriate temperature, gas mixture, pH, glucose concentration, growth factors and nutrients. Growth factors are generally derived from animal blood, such as fetal bovine serum (FBS), fetal bovine calf serum, equine serum or porcine serum. Serum does not contain white blood cells nor red blood cells nor clotting factor (fibrinogens). It still includes the proteins not involved in blood clotting and all the electrolytes, antibodies, antigens, hormones etc. Serum is also believed to protect the cells against physical chocs as it is a viscous liquid. Serum is further known to control the osmotic balance between the cells and the external medium and to control chemical parameters like protease inhibition.

The use of stem cells in veterinary medicine such as equine medicine opens the way to a wide range of therapeutic opportunities by promoting an optimal regeneration of the injured tissue. Indeed, tendinitis and osteoarthritis are very frequent pathologies in equine medicine and unfortunately have a poor prognosis. In fact, musculoskeletal injuries are the most common source of injuries for competing horses. Although it is well known that (almost) adult tissues have some tissue-specific progenitor cells, these are often not sufficient for an efficient repair. Thus, effective regenerative medicine requires an exogenous input of cells in greater numbers than those that are present normally within the tissue. These cells should both be able to repair the lesion as well as to coordinate the healing process.

In human medicine as well, the use of stem cells in therapeutic treatments is the subject of lots of research efforts.

Stem cells show a lot of interesting characteristics. Their production, however, has often been associated with ethical issues. More recently, less controversial sources of pluripotent stem cells have been identified, such as bone marrow, adipose tissue, umbilical cord, umbilical cord blood, Wharton's jelly, peripheral blood and very recently periosteal tissue and muscle. Further, techniques have been developed to induce pluripotency into some specialized cells. Unipotent stem cells have already undergone some significant differentiation and have been identified in relevant tissues of adult beings.

The use of serum in culture medium for eukaryotic cells is nevertheless associated with several issues. One of these results from the fact that the relevant animal needs to be slaughtered to obtain the required serum quantities. Further, more specifically in the case of mammalian cell culture and particularly in the case of mammalian stem cell culture, the presence of animal proteins in the serum entails the presence of xenogenic proteins in the culture medium of cells of a different species. More particularly in medical or veterinary applications, such “contaminations” may lead to sanitary issues, like inflammatory reactions.

Research efforts have focused on the development of different culture media depleted of serum, the media being then enriched by synthetic compounds. Such media are not suited for certain cell types, and more particularly not for stem cell culture. Moreover, they are known to be expensive.

The article by E. N. Clay et al: “Plasma-Alginate Composite Material Provides Improved Mechanical Support for Stem Cell Growth and Delivery”, ACS Applied Bio Material, vol. 2, n° 10, 23 Sep. 2019, pages 4271-4282, discloses 3D cell culture in a medium comprising blood-plasma-hydrogels and plasma-alginate-hydrogels. Platelet-free plasma was prepared, frozen and lyophilized to form a dry powder. A hydrogel was then generated by dissolving the lyophilized plasma in glycine and adding an aqueous calcium chloride solution and water. Mesenchymal stem cells were seeded into such hydrogels and harvested using protease to degrade the clotted blood plasma. The alginate-free plasma gels are considered to show insufficient mechanical properties and the alginate polymer is stated to show a composite structure and to confer improved stability.

J. M. Jalowiec et al: “An In Vitro Investigation of Platelet-Rich Plasma-Gel as a Cell and Growth Factor Delivery Vehicle for Tissue Engineering”, Tissue Engineering. Part C, Methods December 2008, vol. 22, n° 1, 1 Jan. 2016, pages 49-58, discloses 3D cultivation of MSCs in human platelet-rich plasma-gels(hydrogels) incubated in alpha-minimum essential medium containing FBS, bFGF and aminocaproic acid.

K. Satoko et al: “Three-dimensional culture using human plasma-medium gel with fragmin/protamine microparticles for proliferation of various human cells”, Cytotechnology, vol. 66, n° 5, 17 Aug. 2013, pages 791-802, discloses the growing of human cells in human plasma, platelet-rich plasma and platelet-poor plasma, in Dulbecco's modified Eagle medium with fragmin/protamin particles. FIG. 3 shows the culture of human dermal fibroblasts in human plasma-DMEM gels with fragmin/protamin microparticles and fibroblast growth factor, in plasma-DMEM gels with fragmin/protamin microparticles, in plasma-DMEM gels with fibroblast growth factor and in plasma-DMEM gels alone as a control. FIG. 5 shows results obtained with coronary smooth muscle cells, and FIG. 6 shows obtained results with human hematopoietic cells. After the cell culture, the cells were released from the gels by treatment with trypsin.

Similarly, K. Satoko et al.: “Three-dimensional expansion using plasma-medium gel with fragmin/protamine nanoparticles and FGF-2 to stimulate adipose-derived stromal cells and bone marrow-derived mesenchymal stem cells”, Cytotechnology, vol. 66, n° 5, 17 Aug. 2013, pages 791-802, discloses the culture of adipose-derived stromal cells and of bone marrow-derived mesenchymal stem cells in human plasma in Dulbecco's modified Eagle's medium with fragmin/protamin nanoparticles and fibroblast growth factor, in human plasma in Dulbecco's modified Eagle's medium with fragmin/protamin nanoparticles, in human plasma in Dulbecco's modified Eagle's medium with fibroblast growth factor, and in human plasma in Dulbecco's modified Eagle's medium with no supplementation. After the cell culture, the cells were released from the gels by treatment with trypsin.

Trypsin, however is believed to be aggressive towards the cells, more particularly towards the cell walls, and may affect cell viability or other cell quality related features. Trypsin is known to be a cell activating factor, and trypsin treated cells show myeloperoxydase inhibition potential.

B. Lucia et al.: “Human plasma-gels: their preparation and rheological characterization for cell culture applications in tissue engineering”, Journal of the mechanical behavior of biomedical materials, vol. 89, 13 Sep. 2018, pages 107-113, discloses the preparation of human plasma gels making use of transglutaminase, and culture in such plasma gels of human dermal fibroblasts.

B. Carrion et al.: “A Safe and Efficient Method to Retrieve Mesenchymal Stem cells from Three-Dimensional Fibrin Gels”, Tissue Engineering. Part C, Methods December 2008, vol. 20, n° 3, 1 Mar. 2014, pages 252-263, discloses culturing of MSCs in a synthetic matrix of fibrin and thrombin, and harvesting cultured cells by using nattokinase.

There is still a need for alternative methods for stem culture that make use of allogenic growth factors and no xenogeneic or synthetic compounds other than anticoagulation agents used in plasma preparation and which do not require trypsination for cell collection.

More specifically, it would be desirable to use allogenic growth factors derived from the same mammal species as the one from which the stem cells are extracted.

More preferably even, the growth factors should be autologous, meaning derived from the mammal from which the stem cells are extracted for culture.

### SUMMARY OF THE INVENTION

The present invention provides a new cell culture medium, more particularly a 3-D cell culture medium for culturing stem cells, comprising from 5 to 70 vol. % coagulated mammalian blood plasma.

Advantageously, the plasma-based culture medium is prepared by solubilizing blood plasma at the desired concentration in a base medium, for instance DMEM or similar or equivalent base medium known per se in the art of cell culture, which comprises calcium, thereby allowing the blood plasma to coagulate.

The cell culture medium of the present invention advantageously comprises at least 10 vol. % clotted blood plasma, preferably at least 15 vol. %, and at most 60 vol. %, preferably at most 50 vol. %, most preferably about 20 vol. % clotted mammalian blood plasma.

The mammalian plasma may advantageously be plasma from dogs, cats, guinea pigs, cattle, cows, sheep, horses, pigs, primates or humans.

The present invention also provides a new method for culturing stem cells, preferably mesenchymal stem cells, more preferably mammalian mesenchymal stem cells, the method comprising growing the stem cells in a culture medium comprising from 5 to 70 vol. % clotted blood plasma, and releasing the cultured cells without use of trypsin.

Clotted blood plasma constitutes an advantageous 3-D culture medium for growing stem cells, more specifically mammalian mesenchymal stem cells, and further also serves as growth factor(s). No additional growth factor is required and stem cells, more particularly mesenchymal muscle-derived stem cells may grow in all directions, in the absence of any additional growth factor. It has been found that the cultured stem cells grow exclusively within the clotted blood plasma and do not require any other synthetic support from which they would need to be detached for example with trypsin or any other compound having the same or a similar effect.

Blood plasma is derived from blood and contains essentially the same growth factors as serum, but it still includes the clotting factors (fibrinogens). It has now been found that stem cells, and more specifically mesenchymal stem cells, including but not limited to mammalian mesenchymal stem cells, very well grow in clotted plasma and may easily by released therefrom without use of trypsin.

Further, solid clotted plasma constitutes a suitable medium for 3D stem cell culture, which does not require any 3D substrate of some sort. Cell culture hence takes place in an environment close to natural cell environment. In addition, cell growth has proven to be higher than in 2D culture dishes, as the cells may grow at several levels, in all directions.

Blood plasma is relatively common to produce nowadays and may be derived from a living animal, without any significant pain for the animal. As a result, one may culture mammalian mesenchymal stem cells in a culture medium comprising blood plasma from the mammal species or even from the same mammal being the source of the cells to be cultured and further the same as the receptor of the cultured cells. The advantages on the level of contamination will be easily understood by the person skilled in the art.

The preparation of blood plasma is generally known. Blood plasma is separated from blood by centrifugation. An anticoagulant, like citric acid, is generally added to the blood prior to the centrifugation step. For use in accordance with the present invention, the plasma is advantageously prepared with an anticoagulant which preferably is an aqueous composition comprising citric acid and sodium citrate. The composition may further comprise dextrose.

Examples of anticoagulant used in the preparation of blood plasma comprise 5-15 g/L, preferably about 8 g/L citric acid monohydrate, 15-35 g/L, preferably about 22 g/L, sodium citrate dihydrate and 15-35 g/L, preferably about 25 g/L dextrose monohydrate.

Plasma is sterile and generally stored at −20° C. Prior to usage it is thawed and heated at 37° C. It appears that the 3-D culture medium of the invention is particularly well suited for the culturing of stem cells, more specifically stem cells intended to be used in medical or veterinary treatments, as the invention technology allows for use of endogenous growth factors in the culture of stem cells.

Ethical concerns are lowered and contamination of the cell culture by xenogenic or even allogenic proteins and factors is avoided.

It has been found that the invention method is particularly well suited for the culturing of mammalian mesenchymal stem cells generated from muscular tissue, preferably from a muscular microbiopsy as per WO2015/091210. According to this document, microbiopsy specimens are cut in small pieces (size of a tip of scalpel blade) and placed in culture medium, and cultured cells obtained are transferred to a density gradient fractionation, thereby avoiding enzymatic digestion. According to the present invention, the muscular extracts may be cultured in plasma to generate stem cells which may then be released from the plasma based culture medium without use of trypsin, and without going through a density gradient fractionation step, particularly if they are used for differentiation purposes or therapeutic treatments. The stem cell population obtained is homogenous. The present invention hence provides a simple method for stem cell production.

Also, it has been found that no growth factors are required since those contained in the blood plasma and the muscular microbiopsy are sufficient. Should additional growth factors be required for particular reasons, such may obviously be added.

The invention method may further comprise a solubilization step aiming at resolubilizing the solid clotted plasma in order to facilitate harvesting of the proliferated cells. Such solubilization step may be carried out with the assistance of proteolytic enzymes.

Nattokinase has shown to be a suitable enzyme for resolubilizing clotted plasma. Nattokinase may be found in natto, a traditional Japanese food made from soybeans fermented with Bacillus subtilis var. natto. The enzyme is known to have a preventive effect against cardio-vascular pathologies and thrombosis.

In the method of the invention, the nattokinase has shown to be well-performing in degrading the peptide links of clotted plasma, thereby rendering the culture medium liquid again and allowing for harvesting of the cells grown therein. It is obviously important the resolubilization step does not significantly affect the effect or original characteristics of the cells grown in the solubilized substrate; it has been found that cells grown in an invention medium treated with nattokinase maintain their original characteristics. More specifically in the case of culturing mesenchymal stem cells, it has been found that the MSCs recovered after treatment of the 3-D culture medium with nattokinase maintain their adipogenic, osteogenic and chondrogenic potential.

As an alternative, bromelain which is a cysteine protease isolated from pineapple, and tPA (tissue plasminogen activator), a serine protease, have also shown excellent results.

Other techniques are available to resolubilize clotted plasma, like chemical anticoagulants (or blood thinners).

As discussed herein below, it has been found that the stem cells or stem cell population grown in accordance with the present invention surprisingly no longer express marker CD44 and therefore are different from otherwise grown, particularly mammalian muscle-derived, mesenchymal stem cells or stem cell population. They nevertheless preserve their stem cell character, since when resuspended in serum based culture medium, the cells positively express CD44 again.

The present invention further relates to a kit of parts comprising a first container comprising non-coagulated mammalian blood plasma, a second container comprising base medium, and a third container comprising proteolytic enzyme or anticoagulation agent. A user may then easily prepare the invention culture medium by combining the first and second container such as to prepare the desired concentration of blood plasma and thereby allowing the plasma to coagulate. The cells to be cultured or a muscular micropbiopsy may be suspended into the base medium. No additional growth factors are required. One may nevertheless provide for a fourth container comprising bactericide and/or fungicide to be added to the culture medium. Bactericides and/or fungicides may however also be added into the base medium contained in the second container. When the growing time is considered sufficient, the content of the third may be used to solubilize the clotted blood plasma and release the cultured cells.

### BRIEF DESCRIPTION OF THE FIGURES

The present invention is illustrated by the following figures which are to be considered for illustrative purposes only and in no way limit the invention to the embodiments disclosed therein:

FIG. 1: shows microscope pictures (100×enlarged) of cellular growth around explant tissue in invention culture medium, compared to serum based culture medium;

FIG. 2: shows microscope pictures (100×enlarged) of cellular growth in invention culture medium, compared to serum based culture medium;

FIG. 3: shows cell growth curves for MSCs grown in invention culture medium as compared to the same MSCs grown in known serum based culture medium;

FIG. 4: shows growth curves comparing cell growth in different plasma based culture media and to prior art serum based culture medium;

FIG. 5: shows cellular proliferation at different stages in different plasma concentrations;

FIG. 6: is a diagram comparing cell mortality in the case of MSCs grown in invention culture medium and MSCs grown in known serum based culture medium;

FIG. 7: shows microscope pictures at 100× enlargement of MSCs differentiated in adipocytes;

FIG. 8: shows microscope pictures at 100× enlargement of MSCs differentiated in osteocytes;

FIG. 9: shows microscope pictures at 100× enlargement of MSCs differentiated in chondrocytes;

FIG. 10: shows the plasma volume recovered as a function of time, after treatment with nattokinase at ambient temperature and in the fridge;

FIG. 11: shows the volume of liquefied fraction recovered from clotted plasma, as a function of time at different nattokinase concentrations;

FIG. 12: shows the volume of liquefied fraction recovered from clotted plasma, as a function of time at different bromelain concentrations; and

FIG. 13 shows the volume of liquefied fraction recovered from clotted plasma, as a function of time at different concentrations of tPA in comparison to nattokinase.

### DETAILED DESCRIPTION OF THE INVENTION

As used herein, the singular forms “a”, “an”, and “the” include both singular and plural referents unless the context clearly dictates otherwise. By way of example, “a cell” refers to one or more than one cell.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

For general methods relating to the invention, reference is made to well-known textbooks, including, e.g., “Molecular Cloning: A Laboratory Manual, 2nd Ed.” (Sambrook et al., 1989), Animal Cell Culture (R. I. Freshney, ed., 1987), the series Methods in Enzymology (Academic Press), Gene Transfer Vectors for Mammalian Cells (J. M. Miller & M. P. Calos, eds., 1987); “Current Protocols in Molecular Biology and Short Protocols in Molecular Biology, 3rd Ed.” (F. M. Ausubel et al., eds., 1987 & 1995); Recombinant DNA Methodology II (R. Wu ed., Academic Press 1995), incorporated by reference herein.

For further elaboration of general techniques useful in the practice of this invention, the practitioner can refer to standard textbooks and reviews in cell biology, tissue culture, and embryology. Included are “Teratocarcinomas and embryonic stem cells: A practical approach” (E. J. Robertson, ed., IRL Press Ltd. 1987); “Guide to Techniques in Mouse Development” (P. M. Wasserman et al. eds., Academic Press 1993); “Embryonic Stem Cell Differentiation in Vitro” (M. V. Wiles, Meth. Enzymol. 225:900, 1993); “Properties and uses of Embryonic Stem Cells: Prospects for Application to Human Biology and Gene Therapy” (P. D. Rathjen et al., al., 1993). Differentiation of stem cells is reviewed, e.g., in Robertson. 1997. Meth Cell Biol 75: 173; and Pedersen. 1998. Reprod Fertil Dev 10: 31, and Üsas et al., 2011, incorporated by reference herein.

General techniques in cell culture and media collection are outlined in Large Scale Mammalian Cell Culture (Hu et al. 1997. Curr Opin Biotechnol 8: 148); Serum-free Media (K. Kitano. 1991. Biotechnology 17: 73); Large Scale Mammalian Cell Culture (Curr Opin Biotechnol 2: 375, 1991), incorporated by reference herein.

The term “stem cell” refers generally to an unspecialised or relatively less specialised and proliferation-competent cell, which is capable of self-renewal, i.e., can proliferate without differentiation, and which or the progeny of which can give rise to at least one relatively more specialised cell type. The term encompasses stem cells capable of substantially unlimited self-renewal, i.e., wherein the progeny of a stem cell or at least part thereof substantially retains the unspecialised or relatively less specialised phenotype, the differentiation potential, and the proliferation capacity of the mother stem cell, as well as stem cells which display limited self-renewal, i.e., wherein the capacity of the progeny or part thereof for further proliferation and/or differentiation is demonstrably reduced compared to the mother cell. By means of example and not limitation, a stem cell may give rise to descendants that can differentiate along one or more lineages to produce increasingly relatively more specialised cells, wherein such descendants and/or increasingly relatively more specialised cells may themselves be stem cells as defined herein, or even to produce terminally differentiated cells, i.e., fully specialised cells, which may be post-mitotic.

The term “mesenchymal stem cell” or “MSC” as used herein refers to a mammalian adult, mesoderm-derived stem cell that is capable of generating cells of mesenchymal lineages, typically cells of two, preferably of three or more mesenchymal lineages, e.g., osteocytic (bone), chondrocytic (cartilage), myocytic (muscle), tendonocytic (tendon), fibroblastic (connective tissue), adipocytic (fat) and stromogenic (marrow stroma) lineage. Commonly, but without limitation, a cell may be considered MSC if it is capable of forming cells of each of the adipocytic, chondrocytic and osteocytic lineages, using standard, art-accepted differentiation conditions and cellular phenotype evaluation methods, e.g., as described in Pittenger et al. 1999 (Science 284: 143-7) or Barberi et al., 2005 (PLoS Med 2: e161), and Osas et al., 2011.

The term MSC also encompasses the progeny of MSC, e.g., progeny obtained by in vitro or ex vivo propagation of MSC obtained from a biological sample of a subject.

The term “isolating” with reference to a particular component denotes separating that component from at least one other component of a composition from which the former component is thereby “isolated”. The term “isolated” used in relation to any cell, group of cells or a cell population also implies that such cell, group of cells or cell population does not form part of an animal body.

The ISCT determined precisely the qualities cells must possess to be defined as mesenchymal stem cells (MSCs) as follows: the cells must be plastic-adherent, positive for the markers CD73, CD90 and CD105, negative for the markers CD14 (or CD11b), CD34, CD45, CD79a (or CD19) and MHC-II, and must exhibit the ability to differentiate into cells of mesodermal origin such as osteoblasts, chondroblasts and adipocytes (Dominici et al., 2006). The use of other MSC markers such as CD29 or CD44 was also reported (Pittenger et al., 1999). The mammalian muscle-derived MSC cells referred to herein are defined in that they express or co-express (i.e., are positive for) at least the mesenchymal marker CD105, and preferably also one or more of the following markers: CD44 and CD90.

Throughout this specification “co-express” intends to cover the meaning “comprising co-expression of” such that the cells can express other markers in addition to the particular recited markers characterising the cells.

The expression of cell-specific markers can be detected using any suitable immunological technique known in the art, such as immuno-cytochemistry or affinity adsorption, Western blot analysis, FACS, ELISA, etc., or by any suitable biochemical assay of enzyme activity, or by any suitable technique of measuring the quantity of the marker mRNA, e.g., Northern blot, semi-quantitative or quantitative RT-PCR, etc.

The expression of microRNAs may be determined, for example, with an assay for global gene expression (e.g. using a microarray assay for microRNAs expression profiling analysis, a ready-to-use microRNA qPCR plate or RNA sequencing) or by specific detection assays, for example, but not limited to, quantitative PCR, quantitative reverse-transcription (real-time) PCR (qRT-PCR), locked nucleic acid (LNA) real-time PCR, or northern blotting.

Nucleic and amino acid sequence data for marker proteins listed in this disclosure are generally known and can be obtained from public databases such as, among others, from the NIH “Protein Reviews on the Web” database (http://mpr.nci.nih.gov/prow/), the NIH “Entrez Gene” database (http://www.ncbi.nlm.nih.gov/sites/entrez?db=gene) or the Uniprot/Swissprot database (http://www.expasy.org/). Suitable detection reagents and methods for said markers can be designed either on the basis of such sequence information or, more commonly, are available commercially (e.g., labelled monoclonal antibody reagents).

The term “CD105” encompasses the antigen known as CD105, or its synonyms such as endoglin. CD105 is a membrane glycoprotein located on cell surfaces and is a known mesenchymal stem cell marker. As an example, the partial amino acid sequence of the equine CD105 antigen can be found in the Genbank database under accession number AGW16345.1.

The term “CD90” encompasses the antigen CD90, or its synonyms such as Thy-1 membrane glycoprotein. As an example, the amino acid sequence of the equine CD90 antigen can be found in the Genbank database under accession number ACG61223.1.

The term “CD44” encompasses the antigen generally known as CD44, or its synonyms such as Extracellular matrix receptor III, GP90 lymphocyte homing/adhesion receptor, HUTCH-I, Hermes antigen, Hyaluronate receptor, or Phagocytic glycoprotein 1. As an example, the amino acid sequence of the equine CD44 antigen can be found in the Genbank database under accession number CAA47331.1. The expression of CD44 by cells is generally considered as characterizing their potential to attach to a plastic substrate.

Exemplary commercially available antibody reagents for detection of said MSC markers include inter alia monoclonal antibodies anti-CD105-RPE (ABD Serotec), anti-CD44-APC (BD Pharmigen), and anti-CD90 (VMDR). Alternative antibodies that are specifically binding to CD105, CD44, or CD90 can be identified by the person skilled in the art.

The MSCs referred to in the examples express at least one mesenchymal marker chosen from: CD105, CD90 and CD44. MSC cells of such marker profile may also co-express other markers.

MSC cells further display certain morphological features, such as any one or more of adherence to tissue culture plastic; growth in monolayers; and mononuclear ovoid, stellate or spindle shape with round to oval nuclei having prominent nucleoli.

The term “cell population” generally refers to a grouping of cells. A cell population may consist of or may comprise at least a fraction of cells of a common type, or having characteristics in common. Such characteristics may include, without limitation, morphological characteristics, potential for differentiation (e.g., pluripotent, multipotent, unipotent, etc.; e.g., if multipotent or unipotent, ability to differentiate towards specific cell types), or the presence and/or level of one, two, three or more cell-associated markers, e.g., surface antigens. Such characteristics may thus define a cell population or a fraction thereof. Preferably, such a cell population is mesenchymal stem cell population, more preferably a substantially homogenous population of mesenchymal stem cells.

The term “substantially homogeneous” or “substantially pure” population of mesenchymal stem cells denotes a cell population comprising a fraction of MSCs as defined above, wherein said fraction in said cell population is at least 50%, e.g., at least 55%, preferably at least 60%, e.g., at least 65%, more preferably at least 70%, e.g., at least 75%, even more preferably at least 80%, e.g., at least 85%, most preferably at least 90%, e.g., at least 91%, at least 92%, at least 93%, at least 94%, at least 95%, at least 96%, at least 97%, at least 98%, at least 99%, or even close to or equal to 100%.

The expression “density gradient centrifugation” encompasses all types of cell-separation techniques or products encompassing the density-based separation of cells. Non-limiting examples can be density gradient centrifugation in a gradient of sucrose polymer, or colloidal silica. Non-limiting examples of commercially available gradients are: percoll (colloidal silica coated with polyvinylpyrrolidone or silane), ficoll (high molecular weight sucrose-polymers), Ficoll-Paque (Ficoll plus sodium diatrizoate and edetate calcium disodium), buoyant density solution (BDS, comprising colloidal silica), lymphoprep (sodium diatrizoate and polysaccharide), etc. It is clear that the skilled artisan will be able to select suitable gradients to separate the stem cells obtained with the method according to the present invention. Using the methods of the present invention, the mesenchymal stem cells (MSCs) are typically found in the <15%, 15-25%, or 25-35% Percoll density interfaces, after centrifugation at 1250×g (25° C., 20 min). Mesenchymal stem cells co-expressing the desired marker proteins can then be selected, enriched or isolated from the general population of isolated and optionally expanded cells by methods known per se, such as, for example, using fluorescence activated cell-sorting (FACS), magnetic-activated cell sorting (MACS), affinity-based technologies inter alia affinity chromatography, or the preplate technique and combinations thereof. Exemplary methods are reported in Wu et al., 2010 (cf. Cell Tissue Research, June; 340(3):549-67).

Live cells having a desired expression profile are allowed to bind with reagents (most commonly immunological reagents such as, e.g., monoclonal antibodies) specific for the respective markers, wherein said reagents are in turn modified (e.g., by a fluorophore, or by immobilisation on magnetic particles or another type of stationary phase), such as to facilitate for selection or capture of cells bound by said reagents from cells not so bound. For general guidance on these methods, refer inter alia to Flow Cytometry and Cell Sorting, 2nd ed., by Andreas Radbruch (ed.), Springer 1999 (ISBN 3540656308); In Living Color: Protocols in Flow Cytometry and Cell Sorting, 1st ed., by RA Diamond and S Demaggio (eds.), Springer 2000 (ISBN 3540651497); Flow Cytometry Protocols (Methods in Molecular Biology), 2nd ed., by T S Hawley and R G Hawley (eds.), Humana Press 2004 (ISBN 1588292355); Affinity Separations: A Practical Approach, P Matejtschuk (ed.), Oxford University Press, 1997 (ISBN 0199635501); and Dainiak et al. 2007. Adv Biochem Eng Biotechnol 106: 1-18.

For differentiation into e.g. adipocytes, osteocytes and chondrocytes, the MSCs or mesenchymal stem cell populations where cultured in an adequate “differentiation medium”. Said differentiation medium can for example be: for adipogenic differentiation: NH AdipoDiff Medium (Miltenyi Biotec); for chondrogenic differentiation: chondrocyte differentiation medium (NH ChondroDiff Medium; Miltenyi Biotec); for osteogenic differentiation: osteogenic medium (NH OsteoDiff Medium; Miltenyi Biotec). The media listed herein are merely shown as exemplary media, but the skilled person will be able to use any other commercial or specifically developed differentiation medium. Other examples of suitable differentiation media for other cells such as myogenic cells, hematopoetic cells, endothelial cells, neural cells, cardiac cells, or hepatocytes can be done by culturing the MSCs in an adequate myogenic, hematopoetic, endothelial, neuronal, cardial, or hepatocytic differentiation medium respectively, examples of which can e.g. be found in Usas et al., 2011.

The expression “mammal” or “mammalian” refers to all mammals, including, but not limited to, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates, e.g., monkeys and apes, but also humans. Preferred mammals are horses, dogs, or cats.

The term “subject” encompasses all mammals as defined above.

The present invention is further illustrated by the following examples, which do not limit the scope of the invention in any way.

### EXAMPLES

### Materials and Methods

### Example 1: Explant Technique

Microbiopsy procedures were performed on standing, awake horses. Microbiopsy specimens were obtained from triceps brachii muscles (long head, at the intersection of a vertical line extending from the tricipital crest and a line between the scapulo- and radio-humeral joints) of each horse (n=3).

Microbiopsy specimens were collected with a 14-gauge microbiopsy needle and a microbiopsy pistol. Briefly, the sampling site was shaved (one cm square) and aseptically prepared. Each sample (approximately 15 to 20 mg of tissue) was collected at a depth of 5 cm in the long head of the triceps brachii muscle, through a skin incision made with the tip of a scalpel blade nr 11. Closure of the skin incision was not necessary and the whole microbiopsy procedure was completed within 15 minutes.

For experiments in FBS, each sample was placed, immediately after collection, in 6 ml of culture medium composed of DMEM/F12 (Lonza) with 20% fetal bovine serum (FBS—Gibco), 5 ml penicillin (Gibco) (1000 U/ml)-streptomycin (Gibco) (10000 μg/ml), 2.5 ml amphotericin B (250 μg/ml—Gibco). Microbiopsy specimens were kept in culture medium at 4° C. until use.

For experiments in blood plasma, each sample was placed, immediately after collection, in DMEM/F12 base medium comprising 5 ml penicillin (Gibco) (1000 U/ml)-streptomycin (Gibco) (10000 μg/ml), 2.5 ml amphotericin B (250 μg/ml—Gibco). Microbiopsy specimens were kept in the medium at 4° C. until use.

Each microbiopsy specimen was carefully dissected (trying to keep as much as possible only muscular tissue, hence eliminating fibrous tissue parts). Microbiopsy specimens were washed several times in phosphate salt buffer solution (10 mM PBS solution at pH=7.4 containing 137 mM NaCl and 2.7 mM KCl), in order to eliminate red blood cells and other potential impurities. The microbiopsy specimens then were cut in small pieces (size of the tip of the scalpel blade). Culture preparation was performed by use of sterile equipment, under a streamline flow hood. Each piece was placed individually into the 16 central wells of a 24-multiwell dish, each well comprising 400 μl of culture medium as follows:
DMEM F-12 (Lonza) enriched with 20% FBS, further added with penicillin/streptomycin (10000 μg/ml), amphotericin (250 μg/ml); the medium is replaced at regular time intervals in order to provide sufficient nutrients to the cells
DMEM F-12 enriched with 20% blood plasma; the culture medium thus is solid and the proliferated cells will be distributed in the coagulated plasma; there is no possibility to replace the medium but plasma and medium are added in order to provide sufficient nutrients to the cells.

The multi-well dish was then incubated at 37° C. under controlled atmosphere (5% CO2) for several days. A migration of cells occurred.

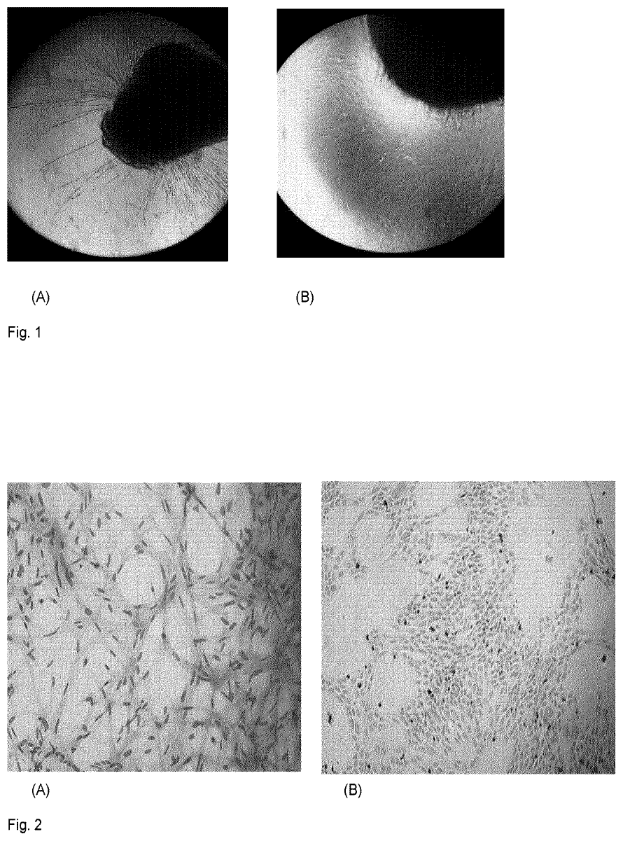

FIG. 1 shows cellular migration out of the muscular tissue for both culture media. A different cellular morphology has been noticed, with cells growing in serum based culture medium showing a fibroblastic morphology and migrating slowly toward the outside of the wells, while the cells growing in plasma based culture medium showing spindle shaped cytoplasmic extensions which appear quickly after start of culturing.

### Example 2: Stem Cell Separation

As far as the wells containing serum based culture medium are concerned, synthetic trypsin (Gibco) was added to the wells at a rate of 150 μl in order to detach the cells from the plastic wells. After 5 minutes at 37° C. double the volume of PBS was added in order to deactivate the enzyme. The content of the wells was agitated by several aspiration-reinjection cycles. The content of the wells was then extracted from each well by a micropipette and transferred to a conical tube. The tubes were then centrifuged at 200×g for 10 minutes. Supernatant was discarded, leaving the cells in the bottom of the tubes. HBSS (Hank's Balanced Salt Solution) medium was then added to the flasks and the solutions were homogenized by several aspiration-reinjection cycles.

As far as the wells loaded with plasma based culture medium are concerned, a proteolytic enzyme first was added in order to liquefy the solid clotted plasma by fibrinolysis. Nattokinase (Nutrisan) in the form of 100 mg capsules was added to a solution of 400 μl EDTA (0.1 M—Sigma-Aldrich) and 40 ml PBS (Gibco). The solution became turbid and was filtered over a 0.2 μm filter, under a hood. The filtered solution was added to each relevant well in order to dissolve the coagulated plasma. The wells were left to stand for approx. 45 minutes in order to optimally liberate the cells from the coagulated mass. The content of the wells was then extracted from each well by a micropipette and transferred to a conical tube. The tubes were then centrifuged at 200×g for 10 minutes. Supernatant was discarded, leaving the cells in the bottom of the tubes. HBSS medium was then added to the tubes and the solutions were homogenized by several aspiration-reinjection cycles.

The cells obtained after culture in serum based culture medium as well as the cells obtained after culture in plasma based culture medium were transferred to a percoll solution (GE Healthcare Bioscience AB) comprising three layers, each having different concentration, and hence a variable density (15-25-35%). The percoll gradient is made up of particles of silica gel covered by polyvinylpyrolidone. After centrifugation at 1250×g for 20 minutes, several phases appeared. The cells moved between two phases and could be harvested easily. The cell fraction between the 15-25% layers was collected, rinsed with HBSS and again centrifuged at 200×g for 10 minutes. The bottom of the tubes was then resuspended in culture medium (serum based: DMEM/F12 (Lonza) with 20% fetal bovine serum (FBS—Gibco), 5 ml penicillin (Gibco) (1000 U/ml)-streptomycin (Gibco) (10000 μg/ml), 2.5 ml amphotericin B (250 μg/ml—Gibco); plasma based DMEM/F12 (Lonza) with 20% plasma, 5 ml penicillin (Gibco) (1000 U/ml)-streptomycin (Gibco) (10000 μg/ml), 2.5 ml amphotericin B (250 μg/ml—Gibco)) and transferred into T-flasks (T-25 Cellstar) incubated at 37° C. under controlled conditions: 5% CO₂. The cells were then passed or frozen, depending on the need for further experiments.

### Example 3: Stem Cell Culture in Order to Establish Growth Curves for MSCs

As far as experiments with plasma based culture medium are concerned, aliquots of 5 ml DMEM-F12 (Lonza) comprising penicillin/streptomycin and amphotericin were deposited in several 25 cm² T-flasks. Thereafter, 200000 MSCs (see Example 2) were added to each flask. Finally 20% blood plasma were added to the culture medium in each flask. The relevant T-flasks were then incubated at 37° C. No further maintenance was required since the cells are dispersed in the plasma.

As far as experiments with serum based culture medium are concerned, the same amount of cells was placed in a growth medium comprising DMEM-F12 (Lonza) and penicillin/streptomycin and amphotericin, and enriched with 20% FBS (Gibco).

After several days, the MSCs were dyed with Diff-Quick in order to show their morphologies. FIG. 2 shows microscope pictures of the cell structures of cells grown in plasma based culture medium (A) compared to cells grown in serum based culture medium (B). It clearly appears that the structures are different and that the cells grown in invention culture medium grow in a cellular network.

A counting of proliferated cells was performed at several stages. Therefore, 3 T-flasks of each condition were evaluated. In order to allow for counting some preparative steps were required.

In the case of plasma based culture medium, nattokinase (2 ml) was added to the culture medium in order to render the coagulated mass liquid. The solution was then transferred to a conical tube for centrifugation at 200×g for 10 minutes. The supernatant was discarded and the bottom redissolved with 5 ml PBS.

In the case of serum based culture medium, the culture medium was extracted from each T-flask and 5 ml PBS were added for rinsing purposes. The PBS was then extracted by aspiration and 1000 trypsin were added in order to detach the cells from the flask surface. The flasks were left to rest for 5 minutes in an incubator in order to allow the enzyme to act. PBS was then added to stop the enzyme activity. The obtained solution was then centrifuged at 200×g for 10 minutes; the supernatant was extracted and 5 ml PBS were added in order to redissolve the content in the bottom.

For cell counting, 10 μl cell suspension samples, from plasma based culture as well as from serum based culture, were placed on a Burker slide. This slide allows for counting the cells; Based on the dilutions that have been made, it is possible to calculate the number of cells in the T-flask. Counting rules pertaining to the relevant device, here a Burker-counter, were applied as instructed by the manufacturer. Trypan blue dye (Hyclone) was added at a rate of ⅕ to differentiate live cells from those that have undergone apoptose, hence providing an evaluation of the cell viability. Proceeding as described at several intervals from initiation of culture provides an evaluation of the cell growth and cell viability.

Further, several plasma parameters have been varied: use of natural plasma having undergone no treatment or modification; use of plasma filtered on a 0.2 μm filter (such filter is believed to filter out large proteins that may negatively affect cell growth); use of plasma centrifuged at 200×g for 10 minutes; use of different plasma concentrations (5, 10, 15, 20, 25 and 30% enrichment of DMEM-F12).

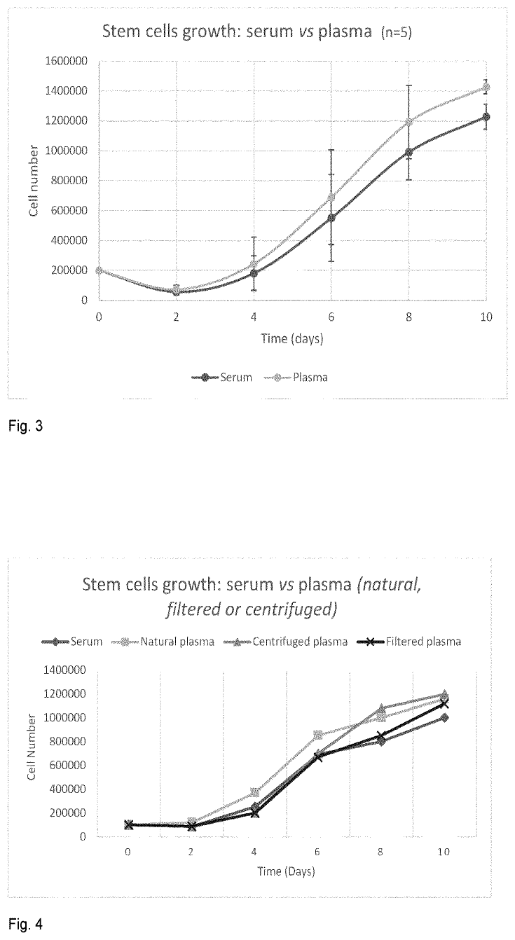

FIG. 3 shows growth curves for MSCs as per Example 2 grown in plasma based culture medium compared to serum based culture medium. Counting was effected at 5 different growing stages.

The results are based on 5 repetitions. Cellular growth was essentially similar for both culture media with a slight decrease after 2 days due to the brutal environmental change to which the cells have to adapt, followed by an exponential growth of the cell numbers and thereafter a slow down due to reduced available space at confluency. It clearly appears that cell growth in invention medium is more efficient than in serum based culture medium.

FIG. 4 shows growth curves comparing cell growth in different plasma based culture media compared to prior art serum based culture medium. One plasma based culture medium comprises natural non-modified plasma, a second plasma based culture medium comprises plasma filtered on a 0.2 μm filter and another plasma based culture medium comprises centrifuged plasma (see here above). The diagram confirms the previous conclusions in relation to FIG. 3. The difference in growth between different plasma based culture media appears statistically insignificant.

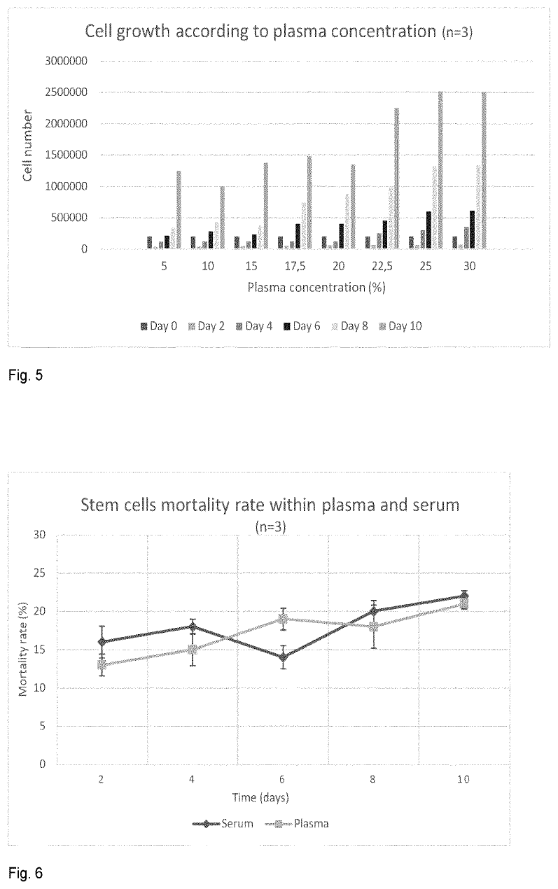

FIG. 5 shows growth of MSCs in plasma based culture media comprising different plasma concentrations. The graph clearly shows that cellular proliferation increases with increasing plasma concentration.

FIG. 6 shows mortality of cells growing in invention medium compared to cells growing in prior art serum based culture medium. It appears that mortality in both situations is similar and remains essentially constant all along the assay manipulations. Overall, mortality stays below 25%.

### Example 4: Evaluation of Functional Properties of the MSCs

It is important to verify that the MSCs obtained in plasma based culture medium retain their functional properties, compared to MSCs cultured in serum based culture medium as control.

The cells to be tested were subjected to flux cytometry in a fluorescence activated cell sorter (FACS®). This technology allows for a non-destructive quantitative analysis of individual cells. The cells pass through a LASER beam. The diffracted light is a measure of the cell dimensions and shape, as well as of characteristics of the cell nucleus. Further, if the cells are prior incubated with fluorescent antibodies and show receptors for said antibodies, the emitted fluorescence is a measure of the quantity of antibodies fixed onto said cells.

The antibodies looked for were those specific for MSCs, i.e. CD44, CD90, CD45 and CMH class II. The assay therefore allows to determine whether the MSCs cultured in invention plasma based culture medium retain their characteristics typical for stem cells. In order to be considered a positive expression, the rate of expression should higher than 70%. For the expression to be considered negative, it should lower than 2%. For MSCs cultured in serum based medium, the expression of CD44 and CD90 should be positive and the expression of CD45 and CMH class II should be negative. The obtained expression levels are shown in the table below:

TABLE

| | % | % | % | % CMH |
|---|---|---|---|---|
| Sample name | CD44 | CD90 | CD45 | class II |
| Sample 1 | 1.5 | 99.9 | 0.6 | 0.9 |
| Sample 2 | 6.6 | 94.4 | 0.1 | 0.1 |

Based on the Table above, the MSCs cultured in invention plasma based culture medium may be viewed as not retaining their full characteristics. It is to be noted that CD44 is a surface protein involved in cellular adhesion. It appears logical that the level of CD44 is reduced when the cells are grown in 3D culture medium. However, when resuspended in serum based culture medium prior to be passed through the FACS, the cells positively expressed CD44 again.

A further characteristic to be determined is the cell potential to differentiate into three possible lineages, osteocytes, chondrocytes and adipocytes. In the osteocyte differentiation, calcium spots were emphasized. In the case of chondrocyte differentiation, proteoglycane production was emphasized and in the case of adipocyte differentiation, lipid production was emphasized. The cells were contacted with relevant differentiation media (Gibco) at a rate of 2 ml per well after they had been transferred into wells of a 6 well dish. The protocol started when the cells had reached confluency. After three weeks, differentiation was evidenced by appropriate dying. Prior to dying, however, the relevant wells need to be prepared as follows. The differentiation medium was extracted from each well and rinsing was performed with PBS. The PBS was then extracted and the cells were fixed with paraformaldehyde at 4%. The wells were allowed to rest for 5 minutes at ambient temperature and thereafter the paraformaldehyde was extracted and the wells rinsed with water. Finally, the relevant dyes were added into the wells in accordance with manufacturer's instructions. In order to evidence adipocytes, Oil REDO (Sigma; 0.5 g in 100 ml isopropanol) was added to the wells; the wells were allowed to stand at ambient temperature for 15 minutes; the dye was extracted and the cells observed under the microscope. In order to show the presence of osteocytes, Alizarin Red (2 g Alizarin Red (Sigma) in 100 ml distilled water, pH adjusted at 4.2 with HCl or NH4OH (10%)) was added to the wells; the wells were allowed to stand at ambient temperature for 15 minutes; the dye was extracted and the cells observed under the microscope. In order to show the presence of chondrocytes, HCL Alcian Blue (Sigma; 3% acetic acid were added to a solution of Alcian Blue 8GX (1 g); pH adjusted to 2.5) was added to the wells; the wells were allowed to stand at ambient temperature for 15 minutes; the dye was extracted and the cells observed under the microscope.

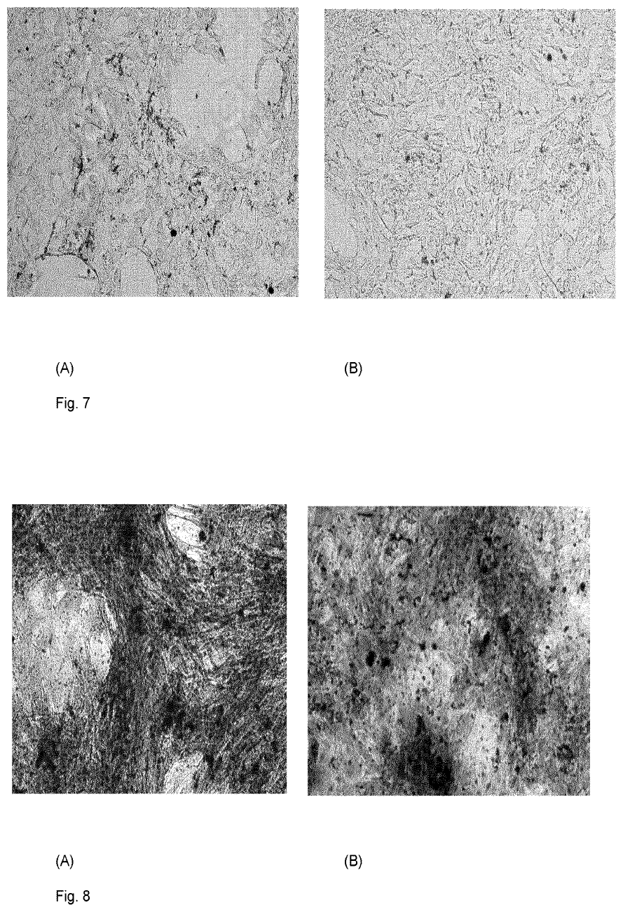

FIG. 7 shows microscope pictures at 100× enlargement of MSCs differentiated in adipocytes. The dark spots represent lipid deposits. Picture A corresponds to cells differentiated after culture in serum based culture medium and picture B corresponds to cells differentiated after culture in plasma based culture medium. Clearly lipid has been generated under both conditions.

FIG. 8 shows microscope pictures at 100× enlargement of MSCs differentiated in osteocytes. Again, dark spots evidence generated calcium deposits. Picture A corresponds to cells differentiated after culture in serum based culture medium and picture B corresponds to cells differentiated after culture in plasma based culture medium. Clearly calcium has been generated under both conditions.

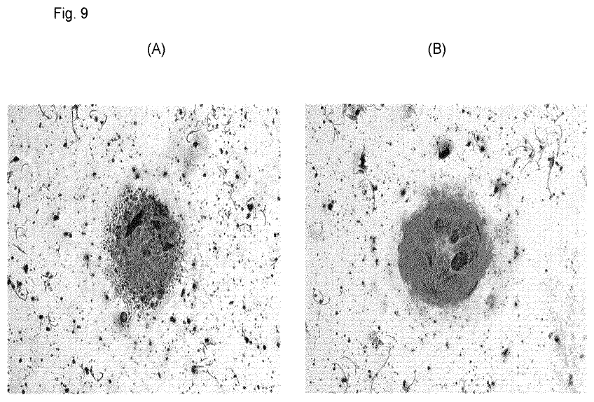

FIG. 9 shows microscope pictures at 100× enlargement of MSCs differentiated in chondrocytes. Again, dark spots evidence generated mucopolysaccharides generated by chondrocytes. Picture A corresponds to cells differentiated after culture in serum based culture medium and picture B corresponds to cells differentiated after culture in plasma based culture medium. Clearly mucopolysaccharides has been generated under both conditions.

### Example 5: Evaluation of the Effect of Nattokinase

The wells of a multi-well dish were filled with approx. 80000 cells and DMEM-F12 culture medium without serum (approx. 500 μl). 100 μl natural unmodified plasma were then added. Thereafter, 400 μl freshly prepared nattokinase (see above) was added into two wells. The multi-well dish was then incubated for 30 minutes. After the 30 minute incubation, the remaining liquid is recovered. The above steps are repeated with two other wells until the plasma fully coagulates and no liquid remains in the wells. The gathered data were compared with that obtained with nattokinase stored in a fridge.

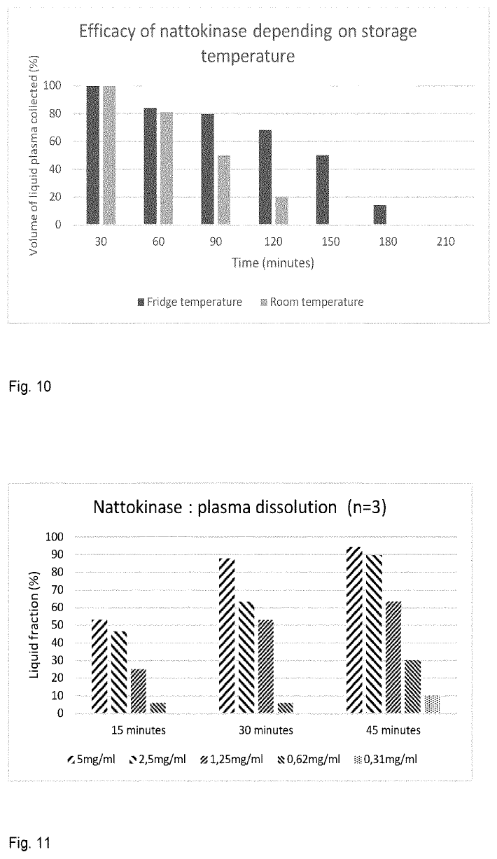

FIG. 10 shows the plasma volume recovered after treatment with nattokinase as a function of time. As can be drawn from the graph, the effect of nattokinase diminishes after 60 minutes. At ambient temperature, the effect diminishes more quickly than the effect measured with nattokinase in a fridge at −4° C.; in latter case the enzyme has an effect over an extended period of time.

### Example 6: Evaluation of the Cell Adhesion Characteristic

MSCs which have been cultured in serum based culture medium as well as those cultured in invention plasma based culture medium were further cultured in a known culture medium in T-flasks. It has been shown that both had retained their characteristic of adhering to a plastic surface.

It has been shown that the invention culture medium allows for rapid cell growth and does not significantly affect the cell's characteristics, more particularly stem cells retain their stem cell characteristics. Further, the method of the invention allows for autologous proliferation of stem cells intended for further treatment of mammalian subjects.

### Example 7: Plasma Dissolution

A comparison of three different enzymes was made in the step of cell release from coagulated blood plasma, different concentrations of the dissolution solutions and incubation times were evaluated as follows:

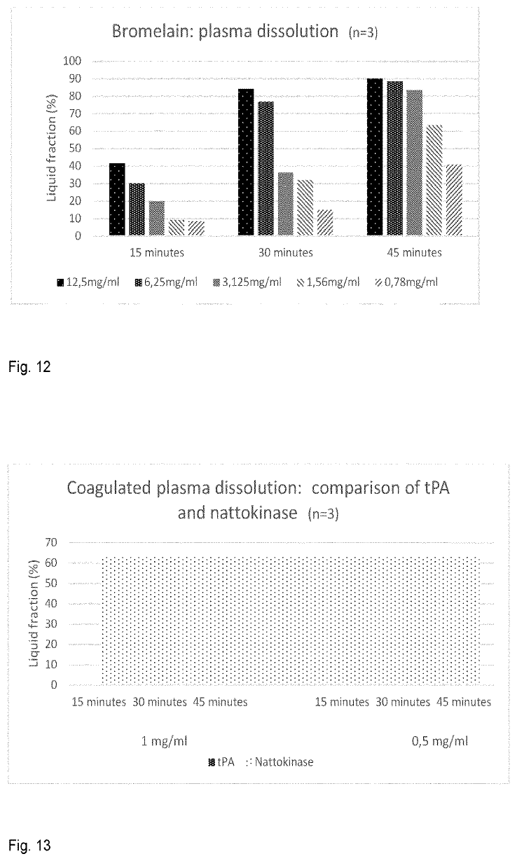

Equine mesenchymal stem cells obtained as per example 1 were previously seeded with 300 μl of plasma medium (DMEM/F12 (Lonza) with 20 vol. % plasma, penicillin (1000 U/ml—Gibco), streptomycin (1000 μg/ml—Gibco), amphoterin B (250 μg/ml—Gibco)) in 24-well culture plates. The dissolution solutions incubated with clotted blood plasma comprising stem cells inside at 37° C. for 15/30/45 minutes. The volume of liquefied fraction was evaluated every 15 minutes. Results are shown in FIGS. 11-13.

The efficacy of proteases seems to be proportional to concentration and incubation time.

A part of the released cells was reseeded in culture plate with plasma. The other part was used for cell viability measurement. To determine cell viability, 10 μl of trypan blue solution are added to 40 μl of cell suspension. Viability is determined by counting under microscope, non-stained cells being considered living cells and blue stained cells being considered dead. The results are shown in the table below:

Viability

| | 1 mg/ml | 0.5 mg/ml |
|---|---|---|
| tPA | 86% | 88% |
| Nattokinase | 91% | 93% |
| Bromelain | 80% | 81% |

As can be seen viability is high after 45 minutes of incubation.

1. A 3-D cell culture medium for the culturing of stem cells, comprising from 5 to 70 vol. % coagulated mammalian blood plasma.

2. The 3-D cell culture medium of claim 1 wherein the mammalian blood plasma serves as the growth factor(s) of the culture medium and 3-D-substrate for 3-D cell growth.

3. The 3-D cell culture medium of claim 1 comprising at least 10 vol. %, preferably at least 15 vol. %, and/or at most 60 vol. %, preferably at most 50 vol. %, most preferably about 20 vol. % coagulated mammalian blood plasma.

4. The 3-D cell culture medium of claim 1 wherein the mammalian plasma is plasma from dogs, cats, guinea pigs, cattle, cows, sheep, horses, pigs, primates or humans.

5. A method for the culturing of stem cells comprising growing the stem cells in a cell culture medium comprising from 5 to 70 vol. % coagulated mammalian blood plasma, and releasing the cultured cells without use of trypsin.

6. The method of claim 5 wherein the blood plasma is blood plasma obtained from blood combined with a citric acid composition.

7. The method of claim 5 wherein the blood plasma is blood plasma obtained from blood combined with a citric acid composition and wherein the citric acid composition comprises citric acid, dextrose and water.

8. The method of claim 5 wherein the stem cells are mammalian muscle-derived mesenchymal cells from a muscular microbiopsy.

9. The method of claim 5 wherein the stem cells are mammalian muscle-derived mesenchymal cells from a muscular microbiopsy and wherein the mammalian mesenchymal stem cells are stem cells from an animal selected from the group consisting of dogs, cats, guinea pigs, cattle, cows, sheep, horses, pigs, primates and humans.

10. The method of claim 5 wherein the stem cells are mammalian muscle-derived mesenchymal cells from a muscular microbiopsy, wherein the mammalian mesenchymal stem cells are stem cells from an animal selected from the group consisting of dogs, cats, guinea pigs, cattle, cows, sheep, horses, pigs, primates and humans and wherein the mammalian plasma is from an animal selected from the group consisting of dogs, cats, guinea pigs, cattle, cows, sheep, horses, pigs, primates and humans.

11. The method of claim 5 further comprising a step of resolubilizing the clotted blood plasma.

12. The method of claim 5 further comprising a step of resolubilizing the clotted blood plasma, wherein the resolubilization is effected with the assistance of a proteolytic enzyme or anticoagulation agent.

13. (canceled)

14. (canceled)

15. A kit comprising a first container comprising non-coagulated mammalian blood plasma, a second container comprising base medium, and a third container comprising proteolytic enzyme or anticoagulation agent.
